(19) United States
(12) Patent Application Publication
(10) Pub. No.: US 2011/0269726 A1
(43) Pub. Date: Nov. 3, 2011

(54) Method of and system for treatment of inflammatory skin conditions

(76) Inventor: Valerie Goldburt, New York, NY (US)

(21) Appl. No.: 12/799,731

(22) Filed: Apr. 30, 2010

(51) Int. Cl. A61K 31/573; A61P 17/00

(52) U.S. Cl. 514/171

(57) ABSTRACT

For treating inflammatory skin conditions a treatment cream including hydrocortisone is applied on a skin; and the applying the treatment cream is combined with treatment of the skin with moisturizer with a moisturizer.

### BACKGROUND OF THE INVENTION

The present invention relates to a method of treatment of inflammatory skin conditions as well as to a system for inflammatory skin conditions.

Methods and systems for treatment of skin defects are known in great varieties. Treatment creams, medications, natural remedies have been used for treating inflammatory skin conditions. It is believed that the existing method and systems for treatment of skin defects can be further improved.

### SUMMARY OF THE INVENTION

Accordingly, it is an object of the present invention to provide a method of and a system for treatment of inflammatory skin conditions, which is a further improvement of the existing methods and systems.

In keeping with these objects and with others which will become apparent hereinafter, one feature of the present invention resides, briefly stated, in a method of inflammatory skin conditions, comprising applying on the skin a treatment cream including hydrocortisone; and combining it with, treating the skin with a moisturizer.

The applying a treatment cream with hydrocortisone, includes applying the treatment cream with hydrocortisone which is contained in the cream in a quantity between 0.75 and 1.25%, for example 1%.

A further feature of the present invention resides in that said applying the treatment cream with hydrocortisone and treating the skin with a moisturizer includes repeating the treating of the skin with moisturizer and the treatment cream with hydrocortisone at least two times a day.

A further feature of the present invention resides in, prior to the moisturizing, treating the skin with a gentle cleanser.

A further feature of the present invention resides in a system for inflammatory skin conditions, comprising a treatment cream including hydrocortisone for applying on the skin; and a moisturizer for treating the skin preliminary to the applying the treatment cream.

A further feature of the present invention resides in said applying a treatment cream contains hydrocortisone in a quantity between 0.75 and 1.25%, for example 1%.

A further feature of the present invention resides that the system also includes a gentle cleanser to treat the skin prior to the moisturizing, treating the skin with a gentle cleanser.

### DESCRIPTION OF THE PREFERRED EMBODIMENTS

The inventive method and system can be used for example for treating such inflammatory skin conditions such as eczema, itching, insect bites, dermatitis, poison ivy rashes, allergies and reactions on the skin.

In accordance with the present invention the following method and system are proposed for treating inflammatory skin conditions.

In the present invention it is suggested to use for inflammatory skin conditions application on a skin a treatment cream including hydrocortisone; and combining it with treating the skin with a moisturizer.

The hydrocortisone in the treatment cream can be within the range of 0.75 to 1.25 weight percent, preferably 1 weight percent.

In accordance with the inventive method and system, before treatment of a skin with the treatment cream containing hydrocortisone the skin is treated with a moisturizer, or the treatment cream and the moisturizer can be applied simultaneously, or it is even possible to apply the moisturizer after applying the hydrocortisone treatment cream.

The treatment cream which contains hydrocortisone can include the following components:

1% hydrocortisone
Hydrocortisone 1
Glycerin 9.0
Soluble animal collagen 8.0
Hydrolyzed elastin 1.0
Triethanolaminme 0.58
Carbomer 940 0.40
Imidazolidynil urea 0.36
Sorbic acid 0.208
Octoxynol-9 0.115
Butylate hydroxytoluene (BHT) 0.203
Sodium hyaluronate 0.011
Purified water 79.2

Moisturizers which are preferable for the inventive method and system can include

### Oil Phase

Carnation mineral oil 19.4
White perfecta petrolatum 5.0
Amerchol L-101 lanolin alcohols extract 5.0
OHlan hydroxylated lanolin 3.0
Arlacel 83 sorbitan sesquioleate 3.0
Oleinate 288 glycerol trioleate 3.0
Ceraphyl 140 decyl oleate 3.0
Sweet almond oil 2.0
Stearic acid 1.5
Amerlate P isopropyl lanolate 1.0
Ceraphyl 424 myristyl myristate 0.1

### Water Phase

Lime water (saturated) 50.0
Ca Cl₂0.48
Distilled water QS 100.0%

Another moisturizer which is preferable for the inventive method and system can include:

### Phase A

Stearic acid 0.5
Isostearic acid 2.5
Robane 2.8
Pur-Cellin oil 1.5
PEG-100 stearate 1.0
Glyceryl stearate 1.0
Amerchol L-101 2.0
Propylparaben 0.15
Cetyl alcohol 1.0
Jojoba oil 2.5
Tenox 4 0.05
Octyl dimethyl 2.0
p-aminobenzoic acid
corn oil 0.4
Wickenol 163 3.0

### Phase B

Deionized water 10.0
Glycerin 4.5
Methylparaben 0.25
Triethanolamine (98%) 0.3
Allantoin 0.2
Panthenol 1.0
Xanthum gum 0.1

### Phase D

2-phenoxyethanol 0.4

### Phase E

FD&C red #4 0.00055
FD&C yellow #5 0.0007

### Phase F

Fragrance 0.25

In accordance with the present invention it is also suggested to treat the skin with a gentle cleanser before treating the skin with a moisturizer. A preferable cleanser which can be used in the inventive method and system includes:
Cetyl alcohol 3.0
Stearyl alcohol 0.5
Poloxamer 123 0.4
Glycerin 2.0
70% sorbitol solution 2.0
steareth-21 0.8
steareth-2 0.2
glyceryl monolaurate 1.0
water QSAD 90.1

In the inventive method the above explained cleaning of the skin with cleanser, moisturizing the skin with a moisturizer, and treating the skin with the treatment cream having hydrocortisone can be performed two times a day, for example in the morning and at night before sleep.

It will be understood that each of the elements described above, or two or more together, may also find a useful application in other types of methods and systems differing from the type described above.

While the invention has been illustrated and described as embodied in method of and system for inflammatory skin conditions, it is not intended to be limited to the details shown, since various modifications and structural changes may be made without departing in any way from the spirit of the present invention.

Without further analysis, the foregoing will so fully reveal the gist of the present invention that others can, be applying current knowledge, readily adapt it for various applications without omitting features that, from the standpoint of prior art, fairly constitute essential characteristics of the generic or specific aspects of this invention.

What is claimed as new and desired to be protected by Letters Patent is set forth in the appended claims.

1. A method of treatment of inflammatory skin conditions, comprising applying on a skin a treatment cream including hydrocortisone; and to the applying the treatment cream, with treating the skin with a moisturizer.

2. A method as defined in claim 1, wherein said applying a treatment cream with hydrocortisone, includes applying the treatment cream with hydrocortisone which is contained in the cream in a quantity between 0.75 and 1.25%.

3. A method as defined in claim 2, wherein said applying the treatment cream with hydrocortisone includes applying the treatment cream with the hydrocortisone in quantity of 1%.

4. A method as defined in claim 1, wherein said applying the treatment cream with hydrocortisone includes applying the treatment cream with hydrocortisone which includes
Hydrocortisone 1
Glycerin 9.0
Soluble animal collagen 8.0
Hydrolyzed elastin 1.0
Triethanolamine 0.58
Carbomer 940 0.40
Imidazollidynil urea 0.36
Sorbic acid 0.208
Octoxynol-9 0.115
Butylate hydroxytoluene (BHT) 0.203
Sodium hyaluronate 0.011
Purified water 79.2

5. A method as defined in claim 1, wherein said applying the treatment cream with hydrocortisone and treating the skin with a moisturizer includes repeating the treating of the skin with moisturizer and applying the treatment cream with hydrocortisone at least two times a day.

6. A method as defined in claim 1; and further comprising, prior to the moisturizing, treating the skin with a gentle cleanser.

7. A method as defined in claim 1, wherein said treating the skin with moisturizer includes treating the skin with a moisturizer selected from the group consisting of
MOISTURIZER 1
Oil phase
Carnation mineral oil 19.4
White perfecta petrolatum 5.0
Amerchol L-101 lanolin alcohols extract 5.0
OHlan hydroxylated lanolin 3.0
Arlacel 83 sorbitan sesquioleate 3.0
Oleinate 288 glycerol trioleate 3.0
Ceraphyl 140 decyl oleate 3.0
Sweet almond oil 2.0
Stearic acid 1.5
Amerlate P isopropyl lanolate 1.0
Ceraphyl 424 myristyl myristate 0.1
Water Phase
Lime water (saturated) 50.0
Ca Cl₂0.48
Distilled water QS 100.0%,
MOISTURIZER 2
Phase A
Stearic acid 0.5
Isostearic acid 2.5
Robane 2.8
Pur-Cellin oil 1.5
PEG-100 stearate 1.0
Glyceryl stearate 1.0
Amerchol L-101 2.0
Propylparaben 0.15
Cetyl alcohol 1.0
Jojoba oil 2.5
Tenox 4 0.05
Octyl dimethyl 2.0
p-aminobenzoic acid
corn oil 0.4
Wickenol 163 3.0
Phase B
Deionized water 10.0
Glycerin 4.5
Methylparaben 0.25
Triethanolamine (98%) 0.3
Allantoin 0.2
Panthenol 1.0
Xanthum gum 0.1
Phase D
2-phenoxyethanol 0.4
Phase E
FD&C red #4 0.00055
FD&C yellow #5 0.0007
Phase F
Fragrance 0.25

8. A method as defined in claim 6, wherein said treating the skin with cleanser includes treating the skin with a cleanser including
Cetyl alcohol 3.0
Stearyl alcohol 0.5
Poloxamer 123 0.4
Glycerin 2.0
70% sorbitol solution 2.0
steareth-21 0.8
steareth-2 0.2
glyceryl monolaurate 1.0
water QSAD 90.1

9. A system for inflammatory skin conditions, comprising a treatment cream including hydrocortisone for applying on a skin; and a moisturizer for applying on a skin combined with the applying the treatment cream.

10. A system as defined in claim 9, wherein said treatment cream with hydrocortisone, includes hydrocortisone which is contained in the cream in a quantity between 0.75 and 1.25%.

11. A system as defined in claim 10, wherein said treatment cream with hydrocortisone includes the hydrocortisone in quantity of 1% of hydrocortisone.

12. A system as defined in claim 9, wherein said treatment cream with hydrocortisone includes
1% hydrocortisone
Hydrocortisone 1
Glycerin 9
Soluble animal collagen 8.0
Hydrolyzed elastin 1.0
Triethanolamine 0.58
Carbomer 940 0.40
Imidazollidynil urea 0.36
Sorbic acid 0.208
Octoxynol-9 0.115
Butylate hydroxytoluene (BHT) 0.203
Sodium hyaluronate 0.011
Purified water 79.2

13. A system as defined in claim 9; and further comprising a gentle cleanser for treating the skin prior to the moisturizing.

14. A system as defined in claim 9, wherein said moisturizer includes is selected from the group consisting of
MOISTURIZER 1
Oil phase
Carnation mineral oil 19.4
White perfecta petrolatum 5.0
Amerchol L-101 lanolin alcohols extract 5.0
OHlan hydroxylated lanolin 3.0
Arlacel 83 sorbitan sesquioleate 3.0
Oleinate 288 glycerol trioleate 3.0
Ceraphyl 140 decyl oleate 3.0
Sweet almond oil 2.0
Stearic acid 1.5
Amerlate P isopropyl lanolate 1.0
Ceraphyl 424 myristyl myristate 0.1
Water Phase
Lime water (saturated) 50.0
Ca Cl₂0.48
Distilled water QS 100.0%, and
MOISTURIZER 2
Phase A
Stearic acid 0.5
Isostearic acid 2.5
Robane 2.8
Pur-Cellin oil 1.5
PEG-100 stearate 1.0
Glyceryl stearate 1.0
Amerchol L-101 2.0
Propylparaben 0.15
Cetyl alcohol 1.0
Jojoba oil 2.5
Tenox 4 0.05
Octyl dimethyl 2.0
p-aminobenzoic acid
corn oil 0.4
Wickenol 163 3.0
Phase B
Deionized water 10.0
Glycerin 4.5
Methylparaben 0.25
Triethanolamine (98%) 0.3
Allantoin 0.2
Panthenol 1.0
Xanthum gum 0.1
Phase D
2-phenoxyethanol 0.4
Phase E
FD&C red #4 0.00055
FD&C yellow #5 0.0007
Phase F
Fragrance 0.25

15. A system as defined in claim 13, wherein said cleanser includes
Cetyl alcohol 3.0
Stearyl alcohol 0.5
Poloxamer 123 0.4
Glycerin 2.0
70% sorbitol solution 2.0
steareth-21 0.8
steareth-2 0.2
glyceryl monolaurate 1.0
water QSAD 90.1,
